(19) United States
(12) Patent Application Publication
(10) Pub. No.: US 2018/0353395 A1
(43) Pub. Date: Dec. 13, 2018

(54) ANTI-UV EMULSIONS STABILIZED WITH LIGNIN AND NANOPARTICLES

(71) Applicants: Centre national de la recherche scientifique, Paris (FR); UNIVERSITE D'ORLEANS, Orleans (FR)

(72) Inventor: Arayik HAMBARDZUMYAN, Paris (FR)

(21) Appl. No.: 15/779,840

(22) PCT Filed: Nov. 28, 2016
(86) PCT No.: PCT/EP2016/078995

§ 371 Date: May 29, 2018

(30) Foreign Application Priority Data
Nov. 30, 2015 (FR) 15 61575

(51) Int. Cl. A61K 8/06; A61Q 17/04; A61K 8/27; A61K 8/29; A61K 8/72; B01F 17/00

(52) U.S. Cl. CPC A61K 8/062; A61Q 17/04; A61K 8/27; B01F 17/0007; A61K 8/72; B01F 17/0085; B01F 17/0028; A61K 8/29

(57) ABSTRACT

An “oil-in-water” emulsion comprising an aqueous phase, an oil phase, and an adsorption layer at the interface of the two phases, said adsorption layer consisting of a lignin matrix into which metal oxide nanoparticles are incorporated, characterised in that the lignin is present in a quantity of less than 2 wt. %, in particular in a quantity of between 0.5 wt. % and 2 wt. %, and in that the metal oxide nanoparticles are present in a quantity less than or equal to 1.5 wt. %, in particular in a quantity of between 0.5 wt. % and 1.5 wt. %, relative to the total weight of the emulsion.

The present invention relates to emulsions, and more specifically to “oil-in-water” type emulsions comprising an adsorption layer between the two phases and offering antioxidant and anti-ultraviolet radiation properties.

### INTRODUCTION

Emulsions are widely produced and used in industry as either materials to be consumed or to be applied to surfaces as agents that are not soluble in water. We find emulsions in pharmaceutics, cosmetics (milks, creams, ointments), cooking (sauces, creams), painting, the road industry, agrochemicals, detergents, rolling mills, the iron and steel industry, and manufacturing of various coatings (printing, adhesives . . . ).

In cosmetics and pharmaceutics, emulsions are an effective way to obtain a harmonious combination of ingredients of different nature and properties, including lipophilic and hydrophilic ingredients, in a homogeneous form that is easy to use.

An emulsion may be defined as an intimate mixture of two immiscible liquid substances, consisting of an aqueous phase and an oily or fatty phase. By mechanical and/or chemical action, the creation of an emulsion makes it possible to mix these two phases, wherein one of the phases is dispersed in the second in the form of small droplets.

There are emulsions of the “oil-in-water” and “water-in-oil” type. Thus, an oil-in-water emulsion (O/W for oil-in-water) is composed of an oily phase dispersed in an aqueous phase. This is a “direct” emulsion.

Conversely, a water-in-oil emulsion (W/O for water-in-oil) is composed of an aqueous phase dispersed in an oily phase. A W/O emulsion is more greasy to the touch, because the feeling corresponds mainly to the nature of the external phase. Such an emulsion is called “inverse”.

From a thermodynamic point of view, emulsions are inherently unstable, particularly over time. The mixture remains stable by virtue of the presence of an ingredient called an emulsifier.

Emulsifiers are most often surfactants. Emulsifiers may be ionic or nonionic in nature. The molecules used may be of natural or synthetic origin.

Some emulsifiers have specific advantages in addition to their stabilizing properties. Thus, certain emulsifiers protect the active ingredients against oxidation and ultraviolet rays, and/or preserves them within emulsions. These emulsifiers, which make it possible to protect the lipophilic active principles contained in the fatty phase, are actively sought. In fact, the oxidative stability of the active molecules in emulsions is a particularly important point in the cosmetics industry.

Free radicals such as hydroxyls, peroxyls and superoxyls are triggers for oxidation of the active molecules. These radicals are generated either in the aqueous phase or at the oil/water interface of the emulsions, under the effect of various external parameters such as light and oxygen. These free radicals can pass through the adsorption layer, and thus initiate oxidative reactions of the products encapsulated in the lipid phase. This oxidation phenomenon is accelerated in the presence of UV rays.

Among the products sensitive to oxidation, mention may be made of vegetable oils, which often constitute a good portion of the fatty phase of cosmetic compositions. When the oils are oxidized, they have a rancid odor and a color change. We may also mention vitamins, which may be degraded following a more or less long exposure to ultraviolet rays.

Thus, emulsions containing fragile lipophilic active principles, in particular those that are degraded/oxidized when exposed to light for too long, must be kept in opaque containers or in smoked glass, and preferably in total darkness, in order to preserve their qualities over time.

This generates a real cost for the manufacturer who must use suitable containers, and represents a disadvantage for the user, who must maintain the emulsion under appropriate conditions in order to maintain the qualities of the active ingredients and oils present in the emulsion.

In order to overcome this need to use an appropriate container, compounds with properties to protect against oxidation and damage caused by ultraviolet (UV) radiation have been identified and integrated into the adsorption layers at the interface of the aqueous and fatty phases.

In fact, the structure of the adsorption layer can very significantly reduce the occurrence of oxidation reactions, by limiting the interactions of the products encapsulated in the lipid phase with the free radicals and the oxygen present in the aqueous phase and/or by absorbing or dispersing the ultraviolet rays.

Thus, this adsorption layer may consist of compounds having antioxidant capabilities and appropriate UV filters, and/or may comprise compounds having the ability to potentiate the effects of other active compounds, thus protecting the lipophilic compounds against oxidation and damage caused by ultraviolet rays.

Among the compounds known for their properties as UV filters, mention may be made of metal oxide nanoparticles, which are conventionally used in cosmetic compositions intended for sun protection. Metal oxide nanoparticles and their use as photoprotective agents are known and described, for example, in patent applications EP 518772 and EP 518773. These nanoparticles make it possible to protect the materials with which they are associated from visible radiation and ultraviolet radiation by absorbing radiation in the wavelength range of 250 nm to 400 nm.

We may also mention lignin, a natural polymer capable of absorbing ultraviolet rays. It has recently been shown that lignin can advantageously be incorporated into UV absorber films, also comprising cellulose nanocrystals (WO 20121156652). Lignin also has antioxidant properties (Pouteau et al., 2003). In addition, lignin acts as an emulsifier for stabilizing emulsions (Rojas et al., 2007).

The patent application US 2002/0182155 describes compositions comprising a combination of particulate fillers (clay, for example) and lignin, which makes it possible to potentiate the effectiveness of compounds capable of absorbing UV radiation, such as nanoparticles of titanium dioxide (TiO₂). Large amounts of titanium dioxide and/or lignin are used in these compositions to achieve a satisfactory Sun Protection Factor (SPF).

The problem to be solved by the present invention is to reduce the amounts of lignin, and especially metal oxide nanoparticles, used in emulsions, while maintaining sufficient protection against the harmful effects of UV rays and free radicals.

In fact, various studies have highlighted the fact that nanoparticles of titanium dioxide and zinc monoxide as used in cosmetics and, in particular, in sun creams, are potentially toxic. At present, the potential adverse effects of these compounds are being actively sought by scientists (Shi et al., 2013, and the AFSSAPS report on nanomaterials in cosmetics, adopted on 15 Mar. 2011).

Nevertheless, there are no compounds with similar UV absorbance capabilities; thus, the research is directed towards the determination of particular conditions which make it possible to reduce the doses of metal oxide nanoparticles used, while maintaining an effective absorbance of UV rays.

### SUMMARY OF THE INVENTION

The present invention relates to an emulsion of the “oil-in-water” type, comprising an adsorption layer ensuring both the stability of the emulsion and protection of the lipophilic active principles against UV radiation and against oxidation agents.

The adsorption layer consists of a lignin matrix, comprising metal oxide nanoparticles in specific proportions, making it possible to optimize the antioxidant and anti-UV properties of this adsorption layer, while minimizing the quantities used of the constituents of this adsorption layer.

More particularly, the present invention relates to an “oil-in-water” type emulsion comprising an aqueous phase, a fatty phase, and an adsorption layer at the interface of the two phases, wherein the adsorption layer consists of a lignin matrix in which are incorporated metal oxide nanoparticles, characterized in that the lignin is present in an amount of less than 2% by weight, and that the metal oxide nanoparticles are present in an amount of less than 1.5% by weight, relative to the total weight of the emulsion.

The present invention relates to the improvement of the properties of this adsorption layer composed of lignin, by incorporation into the lignin matrix of metal oxide nanoparticles, wherein this incorporation, in particular, makes it possible to improve:
the rheological properties of the adsorption layer; and
the capacity of the metal oxide nanoparticles to absorb UV rays, and thus the possibility of reducing their quantity.

### FIGURES

FIG. 1 shows the UV absorbance of the lignin molecule alone. The different wavelengths are presented on the abscissa axis, while the intensity of the absorbance capacity of the lignin molecule is indicated on the ordinate axis.

FIG. 2 shows the UV absorbance of the lignin molecule in aqueous solution, alone or in the presence of different metal oxide nanoparticles: TiO₂ (Eusolex T2000, Eusolex TS, organosolve) and ZnO.

FIG. 3 shows the lignin adsorption kinetics at the fatty phase/aqueous phase interface: wherein the effects of the presence of metal oxide nanoparticles on the surface tension (mN/m) are represented on the abscissa axis as a function of time.

FIG. 4 shows the sun protection factor (SPF) and UVA protection factor (FPUV A) obtained as a function of the concentration of TiO₂ nanoparticles (shown on the x-axis), in the presence of 1.2% of lignin.

### DETAILED DESCRIPTION

The present invention relates to an “oil-in-water” type emulsion comprising an aqueous phase, a fatty phase, and an adsorption layer at the interface of the two phases, wherein the adsorption layer consists of a matrix of lignin in which metal oxide nanoparticles are incorporated, characterized in that the lignin is present in an amount less than or equal to 2% by weight, especially in an amount of 0.5% to 2% by weight, and that the nanoparticles are present in an amount less than or equal to 1.5% by weight, especially in an amount of 0.5% to 1.5% by weight, relative to the total weight of the emulsion.

Of course, the values of the lower and upper limits of the ranges indicated in the present application are within the so-called range.

As indicated above, an emulsion is a dispersion in the form of droplets of two liquids that are immiscible with each other. It consists of an aqueous phase and a fatty phase. An oil-in-water type emulsion is characterized in that the fatty phase is dispersed in the form of droplets in the aqueous phase.

For the purposes of the invention, the term “aqueous phase” is understood to mean the phase which comprises water and/or at least one water-soluble solvent, i.e. a compound that is liquid at ambient temperature and that is miscible with water. The aqueous phase may also comprise any water-soluble or water-dispersible compound compatible with an aqueous phase such as gelling agents, film-forming polymers, thickeners, surfactants and mixtures thereof.

For the purposes of the invention, the fatty phase is understood to mean the phase which includes any liquid fatty substance, generally an oil or a hydrocarbon. This phase may also be referred to as an oily, organic or lipophilic phase, wherein these adjectives are used interchangeably and designate the same phase in the present application.

For the purposes of the invention, the term “adsorption layer” is understood to mean the layer forming at the aqueous phase/fatty phase interface in the emulsion, and making it possible to maintain the stability of the dispersion of the oil droplets in the aqueous phase.

“Ultraviolet filters” or “UV filters” is understood to mean substances that absorb, reflect or disperse such radiation. For the purpose of the present invention, these substances are intended to protect the lipophilic products contained in the fatty phase of the emulsions.

For the purposes of the invention, the expression “lignin matrix” is understood to mean the three-dimensional lignin network that takes shape at the interface of the aqueous and fatty phases, and constitutes the “base” of the adsorption layer around the micro-droplets of the oily phase.

Lignin

Lignin is one of the main components of wood, along with cellulose and hemicellulose. Its main functions are to provide rigidity, waterproofness and resistance to decomposition. All vascular plants, woody and herbaceous, produce lignin.

Lignin is the second most abundant renewable biopolymer on Earth after cellulose. This polymer consists of at least three different types of monomers, the following monolignols:
coumaryl alcohol, called H unit (hydroxyphenyl), without methoxy group;
coniferyl alcohol, called G unit (guaiacyl), with a methoxy group; sinapyl alcohol, called S-unit (syringyl), with two methoxy groups.

The fraction of each monomer varies considerably according to the plant line (gymnosperm, monocotyledonous angiosperm, dicotyledonous angiocepter); the species; the organ; tissue; and, in a general way, the physico-chemical environment in which the plant grows.

There is therefore no single and precise definition of lignin, because of its great variability even within a given species.

Lignin is a polymer that adopts a three-dimensional network structure, also called ‘grid’ or ‘matrix’, wherein the network allows interconnections between the adsorption layers present around each fatty phase droplet.

This matrix offers very interesting mechanical properties, allowing both very good stabilization of the emulsion and good mechanical strength of the adsorption layer.

The lignin used in the emulsion according to the invention is preferably a lignin of natural origin.

Natural lignins are preferably extracted from woody or herbaceous vascular plants by performing acidolysis in an organic solvent. For example, the extraction mode in a dioxane/water medium, in the presence of hydrochloric acid, makes it possible to obtain lignin preparations which are relatively unmodified and low in associated sugars (Monties B, 1988).

According to a particular aspect of the invention, the lignin used in the emulsion according to the invention is derived from a method for extracting cellulose from plants.

According to another aspect of the invention, the lignin used in the emulsion according to the invention is of synthetic origin.

Lignin of synthetic origin, also called ‘DHP’ for ‘dehydropolymer’, may be synthesized in vitro in two stages:
(i) synthesis of coniferyl alcohol or other monolignol and
(ii) polymerization of monolignols in the presence of hydrogen peroxide and peroxidase.

It is to be understood that the lignin used in the emulsion according to the invention may be purified or not, and may have been chemically modified.

The emulsion according to the invention therefore comprises an adsorption layer consisting of a lignin matrix in which specific amounts of metal oxide nanoparticles are incorporated.

Metal Oxide Nanoparticles

The metal oxide nanoparticles are used here as UV filters that are intended to absorb, reflect or disperse the UV radiation.

The metal of this metal oxide is preferably selected from the group consisting of titanium, zinc, cerium, zirconium, iron, copper, and mixtures thereof, preferably from the group consisting of titanium, cerium, zinc, and their mixtures.

In a preferred manner, the metal is titanium.

According to one embodiment, the metal oxide is selected from the group consisting of titanium dioxide (TiO₂), zinc monoxide (ZnO), cerium oxides (Ce₂O₃ and CeO₂), zirconium dioxide (ZrO₂) and copper oxides (CuO and Cu₂O).

According to a preferred aspect of the invention, the metal oxide nanoparticles are nanoparticles of zinc monoxide (ZnO) of CAS number 1314-13-2, or of titanium dioxide (TiO₂) of CAS number 13463-67-7, wherein both types of nanoparticles may be incorporated into cosmetic compositions from a regulatory point of view.

Titanium dioxide nanoparticles are particularly preferred.

It is to be understood that these nanoparticles may be used as commercial products which may comprise up to 30% of other agents or excipients. Advantageously, products having at least 70% of metal oxide nanoparticles are chosen.

By way of information, various commercial compounds comprising nanoparticles that may be used in the emulsions of the invention are listed in the tables below:

TABLE 1

| | | % of metal oxide |
|---|---|---|
| Trade name | INCI name | nanoparticles |
| Standard titanium oxide | Titanium dioxide | 99-100% |
| Eusolex T-Avo ® | Titanium dioxide, Silica | 79.6% |
| Eusolex T -2000 ® | Titanium dioxide, | 80.3% |
| | Alumina, Simethicone | |
| Eusolex T - Eco ® | Titanium dioxide, | 79.6% |
| | Alumina, simethicone | |
| Eusolex T ® | Titanium dioxide, | 77.5% |
| | Simethicone | |
| Eusolex T -S ® | Titanium dioxide, | 73-79% |
| | Alumina, Stearic acid | |

TABLE 2

| | | % of metal oxide |
|---|---|---|
| Trade name | INCI name | nanoparticles |
| Zinc oxide Neutral ® | Zinc oxide | Min 95% |
| Zinc oxide NDM ® | Zinc oxide, | Min 92% |
| | Dimethicone | |
| Z-Cote ® | Zinc oxide | 99-100% |
| Z-Cote Max ® | Zinc oxide, | 96-99% |
| | Diphenyl Capryl | |
| | methicone | |
| Tego-Sun Z500 ® | Zinc oxide | >99.5% |
| Tego-Sun Z800 ® | Zinc oxide, | >94% |
| | Trimetoxycaprylylsilane | |
| Nanox200 ® | Zinc oxide | 99% |

Most preferably, the titanium dioxide nanoparticles used are selected from those of the brand Eusolex T-2000® and Eusolex T-S®, or a mixture of both.

According to another aspect of the invention, a mixture of at least two different types of metal oxide nanoparticles is used. In particular, a mixture of nanoparticles of zinc monoxide and titanium dioxide may be used.

By nanoparticles is meant nanoparticles having a mean diameter of between 2 nm and 100 nm, or between 2 nm and 50 nm, advantageously between 4 nm and 30 nm, preferably between 6 nm and 20 nm, more preferably between 8 nm and 10 nm.

Nanoparticles of metal oxide with a mean diameter of less than 2 nm may be considered. However, when the average diameter is smaller than the pore size of the dermis, the nanoparticles can pass through the skin, which is not a preferred embodiment of the present invention.

The metal oxide nanoparticles are incorporated into the matrix created by the lignin polymer. These nanoparticles are thus “integrated” or “trapped” in this polymer network. Any technique consisting in mixing and homogenizing the two compounds is suitable for the implementation of the invention. It is preferable to obtain good uniformity of the particle distribution in the lignin matrix.

According to a first implementation of the invention, the metal oxide nanoparticles are bonded to the lignin matrix by non-covalent bonds. The nanoparticles are incorporated in the lignin matrix and remain in place by virtue of non-covalent bonds existing between these molecules. Thus, bonds for the assembly of molecules, such as hydrogen bonds, metallic coordination, hydrophobic forces, van der Waals forces, (π-π) stacks, electrostatic and electromagnetic interactions, are indicated.

According to a preferred aspect of the invention, the metal oxide nanoparticles are incorporated into the lignin matrix by sonication.

The technique of sonication is based on the use of ‘sonicators’, which are devices producing ultrasound waves to agitate the molecules and thus promote their homogenization.

According to a second embodiment of the invention, the metal oxide nanoparticles are bonded to the lignin matrix by covalent bonds. A covalent bond is a chemical bond in which two atoms share two electrons of one of their outer layers in order to form a pair of electrons bonding the two atoms.

A covalent interaction between these molecules may be obtained, in particular, when a chemical bond is established between the empty vacant orbital of the metal oxide, and the electronic doublet of oxygen, a very abundant atom in the lignin molecule.

The incorporation of these nanoparticles within the lignin matrix makes it possible to optimize their capacity as UV filters, and thus makes it possible to reduce the quantity of nanoparticles used while retaining satisfactory protection properties against UV rays.

Quantities and Relationships Considered According to the Invention

The present invention relates to an emulsion comprising an adsorption layer consisting of a lignin matrix in which metal oxide nanoparticles are incorporated, characterized in that the lignin is present in an amount less than or equal to 2% by weight, especially in an amount of 0.5% to 2% by weight, and that the nanoparticles are present in an amount less than or equal to 1.5% by weight, especially in an amount 0.5% to 1.5% by weight, relative to the total weight of the emulsion.

As previously mentioned above, it is in fact essential to reduce the amount of these “ancillary” compounds in the emulsions as they are not active compounds, while retaining their stabilizing properties of the emulsion and protecting the active compounds against UV rays and oxidizing agents.

A small amount of metal oxide nanoparticles in the emulsified system is preferable for all the reasons mentioned above; in addition, at high concentrations, it has been observed that the absorptive power of the metal oxide nanoparticles is attenuated.

According to a first aspect, the lignin is present in the emulsion in an amount less than or equal to 2% by weight, relative to the total weight of the emulsion. The lignin may, in particular, be present in an amount of 0.5% to 2% by weight. The lignin may, in particular, be present in an amount of 2, 1.9, 1.8, 1.7, 1.6, 1.5, 1.4, 1.3, 1.2, 1.1, 1, 0.9, 8.0, 7.0, 6.0, or 0.5% by weight, relative to the total weight of the emulsion.

According to a second aspect, the metal oxide nanoparticles are present in the emulsion in an amount less than or equal to 1.5% by weight, relative to the total weight of the emulsion. The nanoparticles may, in particular, be present in an amount of 0.5% to 1.5%. The nanoparticles may, in particular, be present in an amount of 1.5, 1.4, 1.3, 1.2, 1.1, 1, 0.9, 0.8, 0.7, 0.6, or 0.5% by weight, relative to the total weight of the emulsion.

According to one aspect of the invention, in these emulsions, the weight ratio of metal oxide/lignin nanoparticles is less than 1.

More particularly, in these emulsions, the weight ratio of metal oxide/lignin nanoparticles is less than 1, less than 0.9, less than 0.8, less than 0.7, less than 0.6, less than 0.5, less than 0.4, less than 0.3, or less than 0.2.

According to a preferred aspect of the invention, the metal oxide nanoparticles are present in a weight ratio of metal oxide/lignin nanoparticles between 0.1 and 0.8, and more preferably in a weight ratio of metal oxide/lignin nanoparticles of about 0.5.

According to one particular aspect of the invention, the “oil-in-water” type emulsion consists of an aqueous phase, a fatty phase, and an adsorption layer at the interface of the two phases, wherein the adsorption layer consists of a lignin matrix in which metal oxide nanoparticles are incorporated, while the emulsion does not comprise a particulate thickener.

The term “particulate thickener” is understood to mean, in particular, thickeners such as smectite clays, which include montmorillonite, bentonite, bidelite, hectorite, saponite, stevensite, and the like.

It is to be understood that, according to this implementation, the aqueous and fatty phases consist of different lipophilic compounds (in the fatty phase) and hydrophilic compounds (in the aqueous phase) but do not include particulate thickener.

According to a particular aspect of the invention, the fatty phase may represent at least 50% by weight of the emulsion, relative to the total weight of the emulsion. Such an emulsion is referred to as a “concentrated emulsion”.

In particular, the emulsion may comprise at least 50%, 55%, 60%, 65%, 70%, 75%, 80%, or 85% by weight of fatty phase, relative to the total weight of the emulsion.

According to a preferred aspect of the invention, the fatty phase represents between 50 and 70% by weight of the emulsion.

The fatty phase of the emulsion according to the invention comprises at least one fatty substance chosen from fatty substances that are liquid at room temperature (20-25° C.), such as volatile or non-volatile oils of vegetable, mineral or synthetic origin and their mixtures. The fatty phase may also include any usual liposoluble or lipodispersible additive.

For an application in the cosmetic field, these oils are chosen from physiologically acceptable oils.

As oils that may be used in the composition of the invention, mention may be made, for example, of:
hydrocarbon oils of animal origin;
hydrocarbon oils of vegetable origin;
esters and synthetic ethers, especially of fatty acids;
linear or branched hydrocarbons of mineral or synthetic origin, such as paraffin oils, volatile or not, and their derivatives;
partially fluorinated and/or silicone fluorinated oils;
silicone oils;
and their mixtures.

Among the hydrocarbons of the formula CₙHₘ, where n and m are two integer digits, linear, aromatic and cyclic hydrocarbons may be used, regardless of their boiling point.

Essential oils, in particular extracted from plants, are preferred hydrocarbons for the implementation of the invention.

More particularly, hydrocarbons which may be present in the fatty phase of the invention include cyclopentane (C₅H₁₀), hexane (C₆H₁₄), methylcyclohexane (C₇H₁₄), heptane (C₇H₁₆), decane (C₁₀H₂₂), dodecane (C₁₂H₂₆) and toluene (C₇H₅).

The fatty phase in the emulsion according to the invention may be composed of a single oil, in particular a single hydrocarbon, or may also consist of a mixture of two, three or even four different oils.

According to one aspect of the invention, the fatty phase comprises an organic solvent chosen from cyclopentane, hexane, methylcyclohexane, heptane, decane, dodecane and toluene.

According to another aspect of the invention, the fatty phase is mainly, or totally, constituted of an organic solvent chosen from cyclopentane, hexane, methylcyclohexane, heptane, decane, dodecane and toluene.

The aqueous phase of the emulsion according to the invention mainly comprises water and, optionally, one or more compounds miscible with water. The aqueous phase may also include ionic species, pH regulators, and all active preservative and coloring ingredients that are water soluble or water dispersible.

### EXAMPLES

The products used in this study are SIGMA-ALDRICH brand and are used without further purification:

Decane >95%

Lignin: Lignin, alkali low sulfonate content 471003 Aldrich

Nanoparticles of ZnO

Nanoparticles of TiO₂:
a) TiO₂ (Organosolve W877).
b) TiO₂ (Eusolex T-S®)
c) TiO₂ (Eusolex T-2000®)

### Example 1 Preparation of Aqueous Solutions of Lignin and Metal Oxide Nanoparticles

In order to verify the miscibility of these nanoparticles in a polymer matrix of lignin, they were mixed with the aqueous solution of lignin (0.1 g lignin solubilized in 3.5 ml of water) by sonication according to the following conditions: for 7 minutes, 1 second ‘up’, 1 second ‘off’, amplitude 30%, at T°=25° C., with a Vibra cell 75115 sonicator with the trademark BIOBLOCK SCIENTIFIC.

After complete evaporation of the aqueous phase, scanning electron microscopy (SEM) images made it possible to observe that the ZnO and TiO₂ nanoparticles have a high affinity for lignin and that they “penetrate” perfectly into the matrix constituted by this natural polymer.

### Example 2. Preparation of Decane/Water Emulsions Stabilized with Lignin and Metal Oxide Nanoparticles

The emulsions are prepared by stirring the oil and water phases in the presence of the lignin by sonication as described above.

Concentrated emulsions composed of 6.5 decane/3.5 water (v/v) are stabilized with 100 mg of lignin without nanoparticles (1), or with lignin supplemented with 50 mg ZnO (2), 50 mg TiO₂ W877 Organoslove (3), 50 mg TiO₂ (Eusolex TS) (4), or 50 mg TiO₂ (Eusolex T2000) (5).

After complete evaporation of the aqueous and organic phases of these emulsions, the adsorption layer of each of these emulsions was photographed. Scanning electron microscopic images of the adsorption layers make it possible to observe that the adsorption layer has a fairly regular porous structure.

### Example 3. UV Absorbance of the Aqueous Solutions of Lignin in Combination with Different Types of Metal Oxide Nanoparticles

30 g/l aqueous solutions of lignin, with and without ZnO and TiO₂ nanoparticles, are prepared. All the solutions are mixed by sonication for 7 minutes, with pulsations of 0.1 seconds ‘on’, 0.1 seconds ‘off’.

TABLE 3

| | | | | | TiO2 |
|---|---|---|---|---|---|
| | | | TiO2 | TiO2 | (Euso |
| | Lignin | ZnO | (W877) | (Euso TS) | T2000) |
| Compound | (g/l) | (g/l) | (g/l) | (g/l) | (g/l) |
| Sample 1 | 0.055 | | | | |
| (lignin) | | | | | |
| Sample 2 | 0.055 | 0.025 | | | |
| (lignin + ZnO) | | | | | |
| Sample 3 | 0.055 | | 0.025 | | |
| (lignin + TiO2, | | | | | |
| W877) | | | | | |
| Sample 4 | 0.055 | | | 0.025 | |
| (lignin + TiO2, | | | | | |
| Eusolex TS) | | | | | |
| Sample 5 | 0.055 | | | | 0.025 |
| (lignin + TiO2, | | | | | |
| Eus T2000) | | | | | |

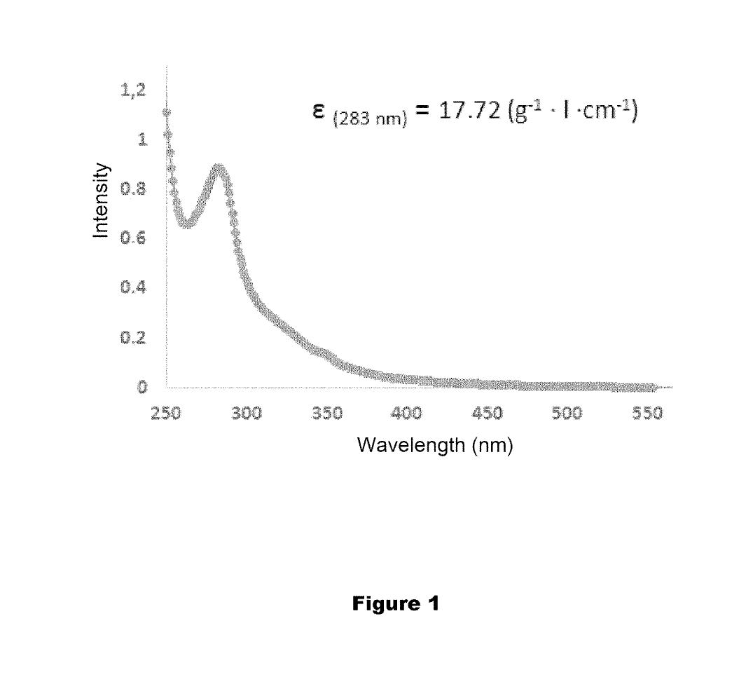

As shown in FIG. 1, lignin alone significantly absorbs rays at the wavelength of 280 nrn.

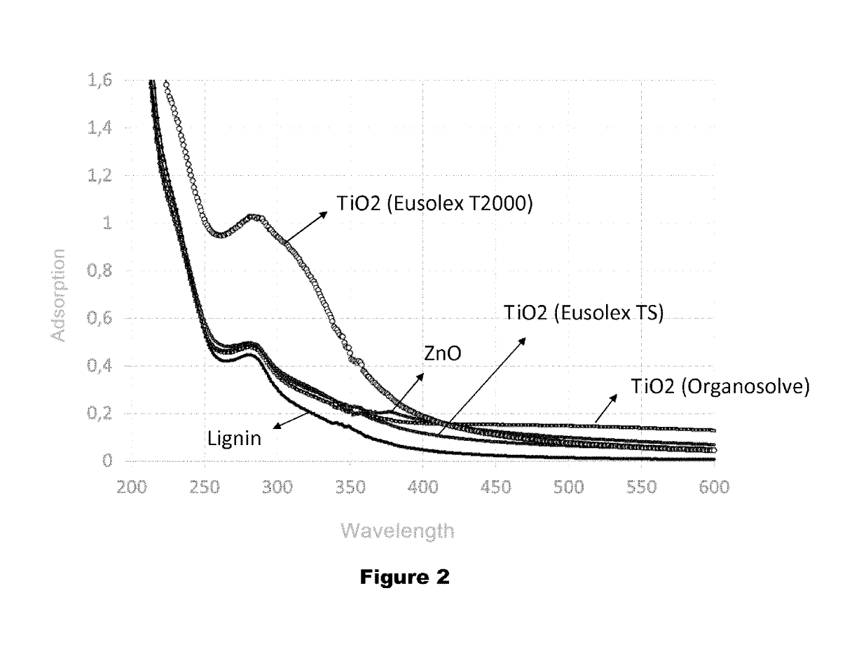

FIG. 2 shows that in the presence of TiO₂ nanoparticles (Eusolex T2000), a significant increase in absorbance intensity of the lignin+nanoparticle combination is observed.

### Example 4. Kinetics of Adsorption of Lignin in Combination with Different Types of Metal Oxide Nanoparticles at the Decane-Water Interface

The main objective of this test is to study the effect of the presence of different nanoparticles (TiO₂ and ZnO) on the rheological properties of the lignin adsorption layer at the decane-water interface.

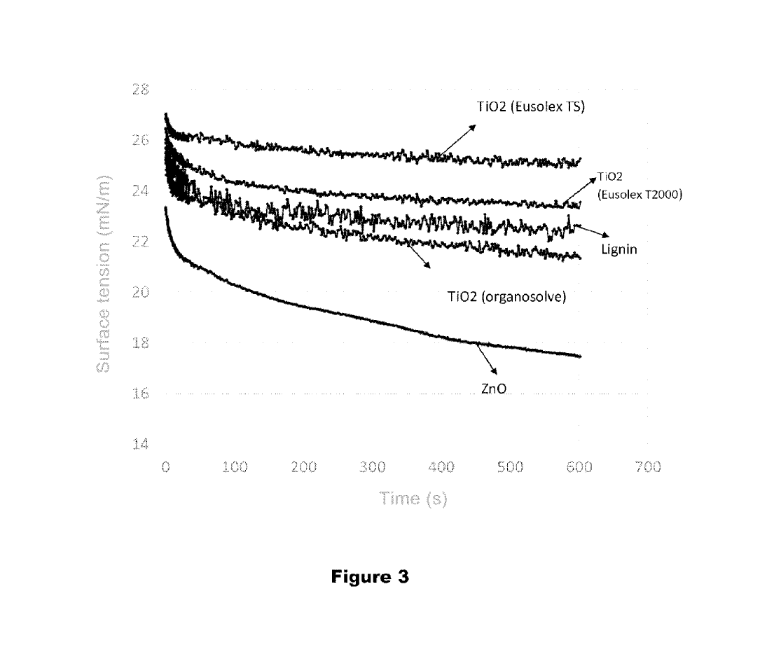

FIG. 3 shows the kinetics of adsorption kinetics of lignin at the decane-water interface with and without nanoparticles.

According to the results obtained, it is found that in the presence of ZnO, the decane-water surface tension is the lowest, whereas with TiO₂ (Eusolex TS) and TiO₂ (Eusolex T2000), the surface properties of lignin are diminished. TiO₂ W877, on the other hand, slightly favors the surface properties of lignin.

The effect of the presence of ZnO and TiO₂ nanoparticles on the physicochemical properties of the lignin adsorption layer at the decane/water interface were collated in the following Table 4:

TABLE 4

| | Surface tension | Surface pressure | Expansion modulus |
|---|---|---|---|
| | (γ) (mN/M) | (π) (mN/M) | (ε) (mN/M) |
| Water/decane | 51.44 | 0 | 1.19 |
| interface | | | |
| Sample 1 | 22.35 | 29.09 | 15.95 |
| (lignin alone) | | | |
| Sample 2 | 17.45 | 33.99 | 50.23 |
| (lignin NPs-1) | | | |
| Sample 3 | 21.38 | 30.06 | 17.96 |
| (lignin NPs-2) | | | |
| Sample 4 | 25.01 | 26.43 | 10.17 |
| (lignin NPs-3) | | | |
| Sample 5 | 23.30 | 28.14 | 14.04 |
| (lignin NPs-4) | | | |

Thus, it is clear that the ZnO nanoparticles increase the expansion modulus of the lignin adsorption layer.

### Example 5. Measurement of Anti-UV Capabilities (SPF Test) of Stabilized Decane/Water Emulsions with Different Lignin Concentrations and Variable Lignin/TiO₂ Ratios

Different samples were tested. The control sample is a formulation outside the invention, comprising 2% of TiO₂ nanoparticles, allowing a comparison of the properties of emulsions 1, 2 and 3 according to the invention. The density of decane is 0.726 g/cm³ at 25° C., which makes it possible to calculate its percentage by weight in the emulsion.

TABLE 5

| | Water | | Decane | | | Lignin | | TiO2 | | Nanoparticles/lignin |
|---|---|---|---|---|---|---|---|---|---|---|
| Sample | ml (g) | % | ml | g | % | g | % | g | % | ratio |
| control | 3.5 | 41.22 | 6.5 | 4.719 | 55.58 | 0.1019 | 1.2 | 0.1698 | 2 | 1.66 |
| 1 | 3.5 | 41.82 | 6.5 | 4.719 | 56.38 | 0.1004 | 1.2 | 0.0502 | 0.6 | 0.5 |
| 2 | 3.5 | 41.95 | 6.5 | 4.719 | 56.55 | 0.1001 | 1.2 | 0.0250 | 0.3 | 0.25 |
| 3 | 3.5 | 42.02 | 6.5 | 4.719 | 56.66 | 0.0999 | 1.2 | 0.099 | 0.12 | 0.1 |

The measurements of the sun protection factor (SPF) and the UV protection factor A are carried out according to conventional techniques well known to persons skilled in the art.

TABLE 6

| Sample | SPF ± DS | FP-UVA ± DS | SPF/FP-UVA ratio |
|---|---|---|---|
| control | 8.09 ± 0.68 | 6.03 ± 0.45 | 1.34 |
| 1 | 4.82 ± 0.66 | 3.98 ± 0.46 | 1.21 |
| 2 | 4.87 ± 0.37 | 3.82 ± 0.24 | 1.27 |
| 3 | 3.44 ± 0.22 | 2.90 ± 0.15 | 1.19 |

It appears that the best SPF is obtained when the concentration of TiO₂ is the greatest (2%) in the control sample.

Nevertheless, and surprisingly, it is observed that the decrease in the TiO₂ content is not accompanied by a similar decrease in the UV protection; in fact:
sample 1 comprises 0.6% of TiO₂, 3.3 times less than in the control sample: nevertheless, the SPF decreases by only 1.678 times, and the FP-UV A by 1.515 times;
sample 3 contains a very low dose of TiO₂: 16.66 times less than in the control sample; nevertheless, the SPF is decreased by only 2.35 times and the FP-UV A by 2.079 times.

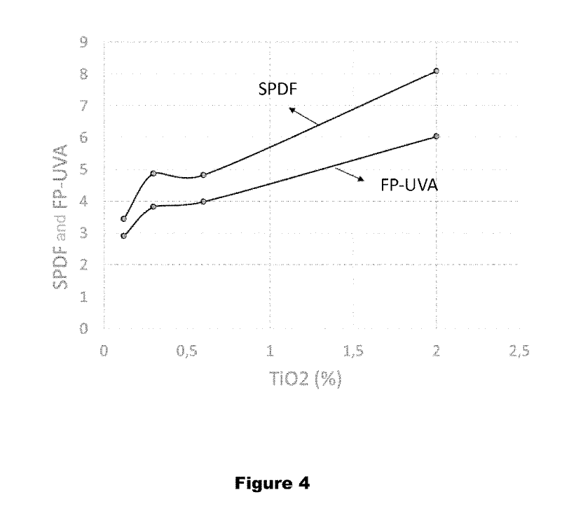

FIG. 4 illustrates this moderate increase in SPF and FPUV A as a function of the concentration of TiO₂ nanoparticles.

Thus, the combination of nanoparticles of titanium dioxide with lignin makes it possible to limit the quantity of nanoparticles used, while maintaining a sun protection factor (SPF) and a protection factor UVA that is sufficient for the protection of the active lipophilic compounds.

### BIBLIOGRAPHIC REFERENCES

EP 518772
EP 518773
WO 2012/156652
US 2002/0182155
C. Pouteau, P. Dole, B. Cathala, L. Averous, N. Boquillon. Antioxidant Properties of Lignin in Poljpropylene, Polymer Dégradation and Stability; 81 (2003) 9-18.
Rojas Orlando.; Bullon Johnny; Ysambertt Fredy; Forgiarini Ana; Salager Jean-L.; Argyropoulos Dimitris. Lignins as emulsion stabilizers. ACS symposium series, 2007, vol. 954, pp. 182-199.
H Shi, R Magaye, V Castranova, J Zhao: Titanium dioxide nanoparticles: a review of current toxicological data. Particle and Fibre Toxicology 2013 10:15
Report adopted by the Cosmetology Commission of Mar. 15, 2011 (AFSSAPS).
Etat des 15 connaissances relatif aux nanoparticules de dioxyde de titane et d'oxyde de zinc dans les produits cosmétiques en termes de pénétration cutanée, degenotoxicite et de cancérogenèse. Saisine 2008 BCT0001
B. Monties. “Preparation of dioxane lignin fractions by acidolysis.” Methods in Enzymology 161 (1988): 31-35.

1. An oil-in-water type emulsion comprising an aqueous phase, a fatty phase, and an adsorption layer at the interface of the two phases, wherein the adsorption layer consists of a lignin matrix in which are incorporated metal oxide nanoparticles, wherein the lignin is present in an amount less than or equal to 2% by weight, and that the metal oxide nanoparticles are present in an amount less than or equal to 1.5% by weight relative to the total weight of the emulsion.

2. The emulsion according to claim 1, wherein the metal oxide nanoparticles are zinc oxide particles or titanium dioxide particles.

3. The emulsion according to claim 1, wherein the metal oxide nanoparticles are bonded to the lignin matrix by non-covalent bonds.

4. The emulsion according to claim 1, wherein the metal oxide nanoparticles are incorporated into the lignin matrix by sonication.

5. The emulsion according to claim 1, wherein the metal oxide nanoparticles are bonded to the lignin matrix by covalent bonds.

6. The emulsion according to claim 1, wherein the metal oxide nanoparticles are present in a weight ratio of nanoparticles/lignin between 0.1 and 0.8.

7. The emulsion according to claim 1, wherein the lignin is of natural origin.

8. The emulsion according to claim 7, wherein the lignin is derived from a method for extracting cellulose from plants.

9. The emulsion according to claim 1, wherein the lignin is of synthetic origin.

10. The emulsion according to claim 1, wherein the fatty phase comprises an organic solvent selected from the group consisting of cyclopentane, hexane, methylcyclohexane, heptane, decane, dodecane and toluene.
